(19) United States
(12) Patent Application Publication
(10) Pub. No.: US 2016/0144137 A1
(43) Pub. Date: May 26, 2016

(54) RETRACTABLE INSULIN INFUSION SYSTEM

(71) Applicant: Paul S. Shapiro, Bloomfield Hills, MI (US)

(72) Inventor: Paul S. Shapiro, Bloomfield Hills, MI (US)

(21) Appl. No.: 14/950,025

(22) Filed: Nov. 24, 2015

Related U.S. Application Data
(60) Provisional application No. 62/083,987, filed on Nov. 25, 2014.
(60) Provisional application No. 62/083,571, filed on Nov. 24, 2014.

(51) Int. Cl. A61M 5/46; A61M 5/142; A61M 5/158

(52) U.S. Cl. CPC A61M 5/46; A61M 5/158; A61M 5/14248; A61M 2005/1586

(57) ABSTRACT

A device for introducing a liquid (e.g., insulin) into tissue of a subject (e.g., a mammal, such as a human) in need of the liquid. The device includes a cannula having a distal tip. A cannula positioner adjustably (and incrementally) controls a position of the cannula after introduction subcutaneously into a subject. A cannula positioner body allows adjustable support of the cannula and the cannula positioner and may be worn by the subject. The cannula positioner includes a cannula position control member that is adapted to be driven (e.g., longitudinally) to advance or retract the cannula so that a subcutaneous position of the distal tip is changeable among a plurality of locations over a period of at least four days, without removal of the cannula from the subject.

### FIELD

The present teachings relate generally to a prolonged duration medical liquid infusion system and more particularly to an insulin infusion system for extended duration subcutaneous dwelling as compared with prior infusion systems.

### BACKGROUND

Individuals afflicted with Type I, and some with Type II, diabetes do not produce insulin (Type I) or are unable to use insulin adequately (Type II). As a result, they are unable to properly control the levels of sugar in their bodies. Treatment of these individuals requires insulin injections to regulate their blood sugar levels appropriately. Prior to the market introduction and commercial production of insulin pumps for delivery, diabetics typically used multiple needle-stick injections of insulin throughout the day in order to control their blood sugar levels. Many continue to favor this practice.

It is understood that the contemporary approach to the use of insulin pumps was as a result of a desire to maintain better control of blood sugar levels and help prevent long-term complications that can occur from diabetes. For a typical insulin pump, the pump is employed to provide a generally continuous “basal” flow of insulin and allow patients to set bolus deliveries of more insulin during meal time. A typical insulin pump system employs a pump that is adapted to cause a specific and predetermined amount of insulin contained within a vessel (e.g., a reservoir containing a volume of insulin (e,g, about 3 milliliters (ml)) to be delivered by way of a tube into a subject by way of a subcutaneously positioned cannula. The cannula is adapted to reside subcutaneously in a subject at a fixed position (i.e., at an initial introductory site) for a period not exceeding three days. An example of a commercially available system is the “Minimed Paradigm” by Medtronic. That system employs consumable componentry, namely the pump connects to a subject using two single-use only disposable products collectively known as the “Silhouette Paradigm Infusion Set”. Another example is the T-slim® insulin pump from Tandem Diabetes Care. Marketed for use with such system is the Comfort™ short infusion set, which includes a flexible cannula that is insertable into a body at a 20-45″ insertion angle.

Use of the above type of system thus requires a subject to employ a single needle stick to introduce the catheter to the subcutaneous initial introductory site. Though such systems have made vast quality of life improvements for many diabetics, because of the reduction of injections and attendant needle sticks, the systems still have certain drawbacks.

For example, because the inserted subcutaneous catheters inject liquid into a single spot under the skin, for some subjects, potential inconveniences may arise. For example, it is possible that subject may experience one or more of a rash, redness, itching, burning, even infection and/or an abscess. In addition, scarring of the area and fatty tissue buildup potentially may occur. When these cannula induced conditions develop, patients may require treatment by a physician. Furthermore, when these conditions arise, it is also believed that the insulin drug may become less effective, potentially resulting in higher than desired blood sugar levels.

Infusion sets on the market have provided improved means of connecting a cannula housing to a delivery device with reduced user discomfort. US Patent Publication No. 2012/0059320 discloses an improved infusion set with an improved means of grasping an introducer and discloses the ability of an adhesive patch to be time sensitive to indicate to the patient when to remove the patch, but does not disclose a means of wearing the patch for a longer duration without the potential for an adverse reaction by the subject. U.S. Pat. No. 8,317,759 discloses an infusion set in which the base portion may be re-oriented on the subject for increased comfort without re-inserting a needle into the subject, but there is no means provided for extending the duration of wear or reducing needle insertions to prevent a subject's adverse reaction to the extended wear.

Though these potential drawbacks are disclosed in the context of insulin infusion, they are not necessarily limited to that environment. The drawbacks may occur in other systems for delivery of liquids to a subject, particularly when a cannula is to reside in a fixed subcutaneous position for an extended period of time.

As a result of the above, there is a need for an improved system for subcutaneous liquid delivery into a subject, such as a mammalian subject (e.g., a human). There is also a need for a system that requires less frequent needle sticks, that can employ a single cannula having at least one portion that resides subcutaneously for a prolonged duration without complete removal (e.g., for a period of at least 4 days, 7 days, 10 days, 14 days or longer), and with a substantially reduced likelihood of the above-noted cannula induced conditions arising.

Examples of patents that may relate to the present teachings include U.S. Pat. Nos. 6,056,718 and 7,303,543, both incorporated by reference for all purposes. Additionally, U.S. Provisional Application Nos. 62/083,571 and 62/083,987 are incorporated by reference for all purposes, specifically all of the combinations set forth in the claims.

### SUMMARY

The present teachings meet one or more of the above needs by providing a device for introducing a liquid (e.g., insulin) into tissue of a subject (e.g., a mammal, such as a human) in need of the liquid. The device includes a cannula having a distal tip. A cannula positioner is employed and is adapted for adjustably (and incrementally) controlling (e.g., manually, in an automated manner, or a combination thereof) a position (e.g., a longitudinal position) of the cannula after introduction subcutaneously into a subject (e.g., a mammal, such as a human). A cannula positioner body (which may include a septum through which an infusion needle penetrates for delivering insulin) is configured to allow adjustable support of the cannula and the cannula positioner and being wearable (e.g., by an adhesive patch connected to the cannula positioner body) by the subject (e.g., the mammal such as the human). Desirably, the cannula positioner includes a cannula position control member that is adapted to be driven (e.g., longitudinally) manually, in an automated manner or both, to incrementally advance or retract the cannula so that a subcutaneous position of the distal tip is changeable a plurality of times over a period of at least four days, without removal of the cannula from the mammal.

As will be seen variations of the above concept are envisioned and will be described in the following. Further, it will be seen that the above may be included as part of a liquid delivery kit that may include one or more of a pump, a liquid delivery tube (which may include a suitable connector for attachment to the device of the teachings herein), a reservoir containing a liquid (e.g., insulin) for introduction to a subject in need of the liquid. Any such kit may also include one or more accessories, such as a skin preparation composition, a skin barrier, an adhesive remover, a topical medicament, a tape, a dressing, a casing, or any other accessory for enhancing the comfort and/or longevity of wear for the subject.

The teachings also contemplate methods of making and/or using the devices of the present teachings. For example one such method may include a step of introducing the cannula subcutaneously into a subject to locate the distal tip at an initial introduction tip location. The method may include a step of retaining the cannula subcutaneously while simultaneously adjusting (one or a plurality of times) the location of the distal tip (e.g., by a predetermined fixed incremental distance (e.g., about 1 millimeter (mm), 2 mm, 3 mm, 4 mm, 5 mm or more), which may be in a longitudinal direction away from the initial introduction tip location). The method may employ a plurality of steps of adjusting the location of the distal tip. Thus, the method may be performed in a manner that accommodates at least 2, 3, 4, 5, 6, 7 or more distal tip position changes to take place over a period of at least 4, 7, 10, 14 days or longer, without removal of the distal tip from the subject (e.g., without removal of the distal tip from a subcutaneous location within the subject (e.g mammal, such as a human)).

It is seen that by employment of the teachings it is possible to reduce needle sticks that a person in need of a needle delivered fluid is required to be subjected over a fixed period of time. This is particularly attractive for use by diabetics in need of insulin, and can allow such individuals to employ a single insulin infusion set for a prolonged period, with fewer potential needle induced conditions arising.

### BRIEF DESCRIPTION OF THE DRAWINGS

FIG. 1 is a plan partially schematic view of an illustrative infusion set and system.

FIGS. 2a and 2b illustrate a track for a position control member which allows the position control member to advance in one direction and prevents motion in an opposing direction.

FIGS. 3a and 3b illustrate a rotary position control member which is adapted to be rotated in a manner which incrementally translates a cannula.

### DETAILED DESCRIPTION

The explanations and illustrations presented herein are intended to acquaint others skilled in the art with the present teachings, its principles, and its practical application. The specific embodiments of the present teachings as set forth are not intended as being exhaustive or limiting of the present teachings. The scope of the present teachings should be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The present teachings are premised upon a recognition of a need for improved subcutaneous liquid delivery systems, as well as a unique device and method for meeting such need. The liquid may be a liquid for a subject in need of treatment by the liquid. The liquid may be one including a medicine. The liquid may be insulin.

In one respect the teachings herein are directed to a device for introducing liquid into tissue of a subject (e.g., a mammal, such as a human). The device includes a cannula having a distal tip. A cannula positioner may employed. The cannula positioner may be adapted for adjustably (and incrementally) controlling (e.g., manually, in an automated manner, or a combination thereof) a position (e.g., a longitudinal position) of the cannula after introduction subcutaneously into a subject (e.g., a mammal, such as a human). A cannula positioner body (which may be in the form of a cannula housing, and which may include a septum and one or more features for facilitating a mechanical interlock) may be configured to allow adjustable support of the cannula and the cannula positioner and be wearable (e.g., by an adhesive patch connected to the cannula positioner body) by the subject (e.g., the mammal such as the human). Desirably, the cannula positioner includes a cannula position control member that is adapted to be driven (e.g., longitudinally) manually, in an automated manner or both, to incrementally advance or retract the cannula so that a subcutaneous position of the distal tip is changeable a plurality of times over a period of at least four days, without removal of the cannula from the subject (e.g., the mammal such as a human).

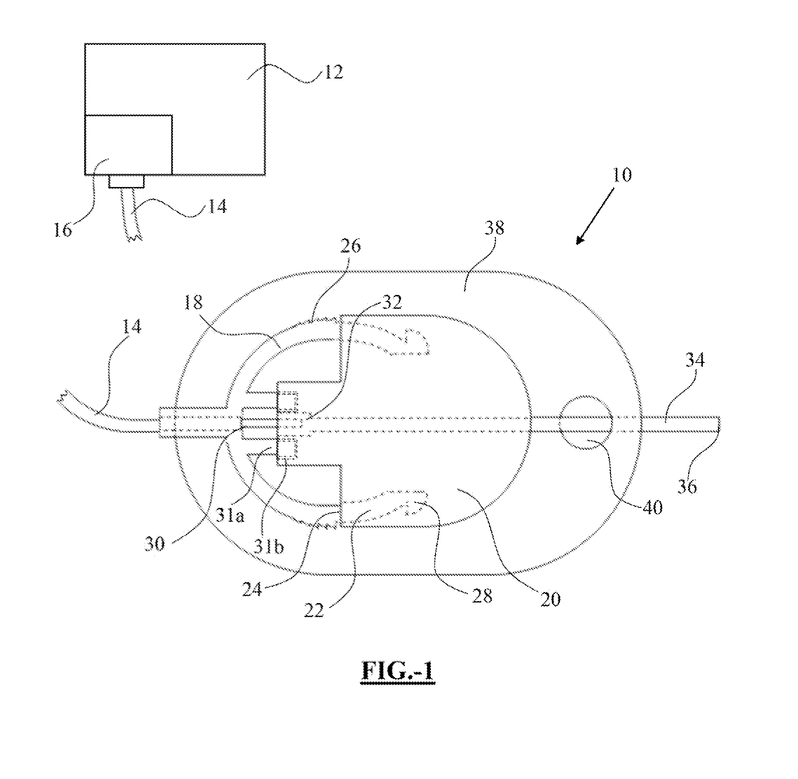

The cannula positioner body may be in fluid communication (e.g., via a supply line, such as a plastic tubing that is sufficiently flexible that a subject wearing it will not rupture the tubing or induce tubing kinks that would disrupt fluid flow) with a liquid supply. The device of the teachings may include an interface structure (e.g., a removable infusion hub) that may be operably connectable (e.g., by way of a mechanical interlock) with and disconnectable from a liquid supply device (e.g., an insulin pump device) in a manner for introducing a liquid (e.g., insulin), when connected with the liquid supply device (e.g., insulin pump device), from a supply of liquid (e.g., insulin) associated with (e.g., at least partially carried on or by) one or both of the liquid supply device (e.g., insulin pump device; which may include suitable delivery tubing from a pumping unit), or the cannula positioner body to the cannula, and into the subject (e.g., mammal, such as a human). Examples of teachings that illustrate how a device in accordance with the present teachings, may interconnect with a removable infusion hub can be seen in the depictions (e.g., FIGS. 6, 7 and 10, and associated written description) of U.S. Pat. No. 6,056,718, incorporated by reference for all purposes; see also FIGS. 1, 1a, 2, and 6-12, and associated written description of U.S. Pat. No. 7,303,543 incorporated by reference for all purposes.

The liquid supply device may be coupled in pumping relation with the liquid supply. For example, the liquid supply may substantially adjoin a pump, and may be remotely located from the cannula positioner body of the device. It is also possible that the cannula positioner body of the device may carry a source of liquid (e.g., insulin) in liquid communication with the cannula. For example, the cannula positioner body of the device may support a liquid supply and an adjoining pump, and the resulting system may be free of any liquid delivery tubing.

It is also possible that the device of the present teachings is employed in a system that is controlled (e.g., in an open loop or a closed loop system) for causing automatic delivery of a liquid to a subject in need (e.g., a mammal, such as a human in need) upon sensing that such subject is in need. For instance, a glucose monitor may sense that a subject (e.g., a mammal such as a human) is in need of insulin, and will issue a signal to cause an insulin pump to deliver insulin to such subject by way of the device of the teachings. Thus, it is appreciated that the device of the teachings may include a cannula positioner body that may (or may not) carry a pump that is operative to cause delivery of insulin to the cannula for introduction into the mammal (e.g., upon an automated sensing of a need for insulin)).

The device of the teachings may employ a cannula that has a length of about 20 to about 50 mm, about 25 to about 45 mm, about 30 to about 40 mm. The cannula may be subcutaneously insertable for at least about 50%, 60%, 70%, 80% or longer, of the entire length of the cannula. The cannula may be subcutaneously insertable to a first subcutaneous position to which liquid (e.g., insulin) is delivered (e.g., through the distal tip of the cannula). The cannula may be positioned and operable subcutaneously for periodic and incremental (e.g., in approximately equal intervals, such as in an amount of about 1, 2, 3, 4, 5 mm or more) removal (e.g., longitudinal retraction) from its first subcutaneous position (i.e., the position at which the cannula is inserted for purposes of the first fluid (e.g., insulin infusion)).

The device may be such that the cannula positioner, the cannula positioner body or both may include a structure defining a guide track along which the cannula positioner is translatable for causing the cannula to be repositioned subcutaneously relative to a first subcutaneous position to one or more second subcutaneous positions (e.g., after at least one delivery of liquid (e.g., insulin) to the site of the cannula tip at the first subcutaneous position and after a predetermined periodic interval (e.g., about 12 hours, 24 hours 36 hours, 48 hours, 60 hours 72 hours or longer) has elapsed).

By way of example, the cannula positioner may include a knob (which may include a serrated edge or other structure for facilitating dexterity by a subject or other individual adjusting position). The cannula positioner may be configured to be manually actuated (e.g., linearly, arcuately, rotationally or both) to cause the cannula to be repositioned subcutaneously relative to a first subcutaneous position to one or more second subcutaneous positions (e.g., after at least one delivery of liquid (e.g., insulin) to the site of the cannula tip at the first subcutaneous position and after a predetermined periodic interval has elapsed). Such repositioning may be in one or more fixed and/or predetermined increments.

Thus, it is envisioned that a manual actuation of the knob may be in a rotary direction that causes at least a portion of the cannula positioner to translate the cannula generally longitudinally between two or more subcutaneous positions (e.g., from a first subcutaneous position to one or more second subcutaneous positions, such as,after at least one delivery of liquid (e.g., insulin) to the site of the cannula tip at the first subcutaneous position and after a predetermined periodic interval has elapsed).

It is also envisioned that a manual actuation of the knob may be in a longitudinal axial direction that causes at least a portion of the cannula positioner to translate the cannula generally longitudinally between two or more subcutaneous positions (e.g., from a first subcutaneous position to one or more second subcutaneous positions, such as after at least one delivery of liquid (e.g., insulin) to the site of the cannula tip at the first subcutaneous position and after a predetermined periodic interval has elapsed).

The cannula positioner may include a direction restrictor that is configured and operable to permit the cannula to move from a first subcutaneous position to one or more second subcutaneous positions, while preventing the cannula from returning toward the first subcutaneous position from one or more second subcutaneous positions. By way of example, the direction restrictor may include one or more mechanical features that permit travel (e.g., incremental travel) in one direction but prevent travel in an opposite direction (e.g., a gear tooth and a co-acting pawl and/or gear tooth, or some other ratchet action device).

The cannula positioner body may be adapted to be mechanically interlocked with a conduit associated with a liquid (e.g., insulin) supply control unit. For example, a fluid supply line (e.g., tubing) may have a suitable supply line connector, such as a mechanical connector (e.g., one that has laterally flanking hinged grip arms, with a detent that co-acts with a receptacle defined within the cannula positioner body). The cannula positioner body may include one or more male or female portions that matingly join with a respective female or male portions. The cannula positioner body may include one or more receptacles into which liquid is introduced by way of a needle or other portion (e.g., a terminal portion) of the conduit or fluid supply line. For example a needle may be housed in the supply line connector.

The teachings also include use of a device of the teachings for administering liquid (e.g., insulin) to a mammal in need of the administered liquid. For example, there is envisioned a method of using the device of any of the preceding claims for administering a liquid (e.g., insulin) to a subject in need of liquid (e.g., insulin) (e.g., a human), comprising inserting the device into a mammal to locate the cannula in a first subcutaneous position, and periodically and incrementally (e.g., in fixed predetermined increments) changing the location of the cannula relative to the first subcutaneous position, while maintaining the cannula within the mammal continuously for a period of at least four consecutive days (e.g., at least seven consecutive days, at least 14 consecutive days or even at least 28 consecutive days). To illustrate in more detail, the teachings envision a method by which there is a step of introducing the cannula subcutaneously into a subject to locate the distal tip at an initial introduction tip location. The method may include a step of retaining the cannula (e.g., at least the distal tip of the cannula) subcutaneously while simultaneously adjusting (one or a plurality of times) the location of the distal tip (e.g., by a predetermined fixed incremental distance (e.g., about 1 millimeter (mm), 2 mm, 3 mm, 4 mm, 5 mm, 7 mm, 10 mm or more), which may be in a longitudinal direction away from the initial introduction tip location). The method may employ a plurality of steps of adjusting the location of the distal tip. Thus, the method may be performed in a manner that accommodates at least 2, 3, 4, 5, 6, 7 or more distal tip position changes to take place over a period of at least 4, 7, 10, 14 days or longer, without removal of the distal tip from the subject (e.g., without removal of the distal tip from a subcutaneous location within the subject (e.g., mammal, such as a human). A method of the teachings may also contemplate instructing a person or subject to administer a liquid to a subject using a device, method or both, in accordance with the present teachings.

The teachings also envision kits that include the device of the present teachings and one or more additional fluid delivery products, such as a tubing, a pump, a liquid supply (e.g., a reservoir of insulin), a subject condition monitor (e.g., a glucose monitor), or any combination thereof. Examples of commercially available insulin infusion systems for which the present teachings may be applicable include, without limitation, an insulin pump system named the MINIMED® insulin pump system available from Medtronic, Inc. The pump system may be provided with a real time glucose monitoring function, a remote glucose monitoring system, a combination thereof, or no glucose monitoring system.

With reference to the drawings of FIGS. 1-3, there are illustrated two examples (without limitation) of embodiments in accordance with the present teachings.

Turning now to FIG. 1, there is shown an example of a liquid infusion system of the present teachings, in the particular context of an insulin infusion system 10. An optional glucose monitor is not shown, but is contemplated as within the embodiments of the general and specific teachings herein. The system includes an infusion pump 12. A supply line (e.g., plastic tubing) 14 connects at one end with a reservoir 16 of insulin associated with the pump 12. The supply line 14 connects with a line connector 18 at another end of the supply line. The line connector 18 includes a structure for creating a mechanical interlock between the connector 18 and a cannula positioner body 20. For example, it includes a pair of male projections 22 that penetrate into opposing slots 24 and engage in an internal structure (not shown, but which may include a forward facing wall (i.e., a wall having a face in the direction in which the cannula enters a body)) defined in the cannula positioner body 20. The line connector 18 includes a gripping portion 26 that includes a free end 28 having a bulbous portion latch finger that can be squeezed together for mating engagement with a suitable recess in the cannula positioner body 20 so that an interference fit can be achieved (e.g., as between the bulbous portion latch finger and the forward facing wall). A needle 30 associated with the line connector 18 is positioned upon connection of the line connector 18 with the cannula positioner body 20 to deliver insulin from the reservoir 16 into a receptacle 32 of the cannula positioner body 20, which is in turn in fluid communication with a cannula 34 having a distal tip 36. The cannula 34 is engaged in the cannula positioner body 20 (e.g., friction fit, interference fit). An adhesive patch 38 supports the cannula positioner body 20, and attaches (e.g., adhesively) to a subject. The patch may include a throughhole 40 through which the cannula penetrates on its way toward penetrating the skin of a subject. There may be one or more projecting prongs 31a that matingly insert within sockets 31b defined in the cannula positioner body 20. The prongs may be employed such as for guidance and/or stabilization.

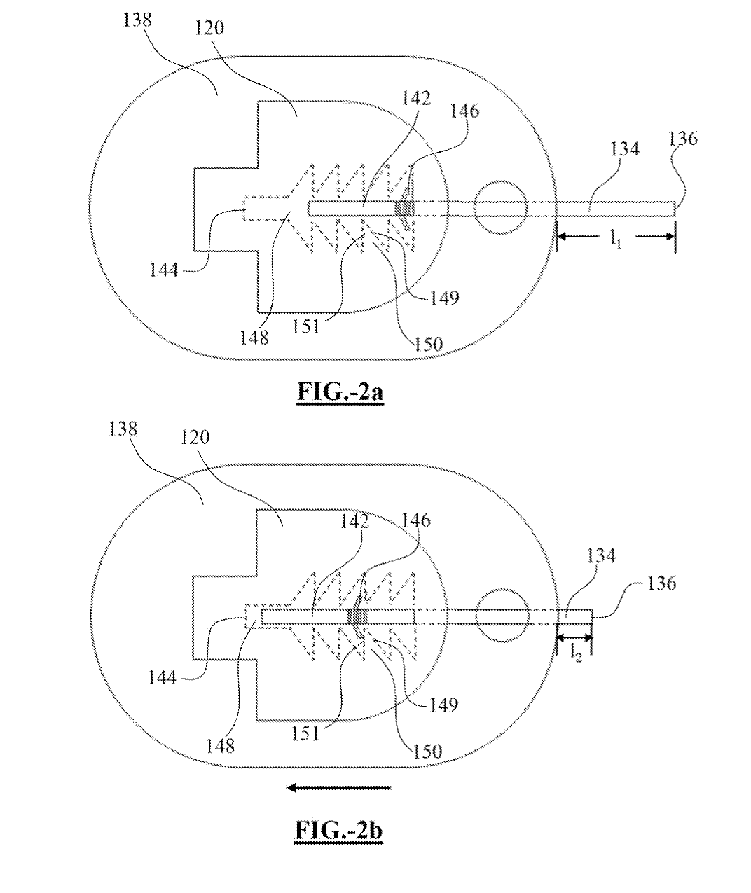

Turning to FIG. 2a, there is seen an example of an embodiment in which a cannula positioner body 120 is adapted to permit longitudinal adjustment of a cannula 134 after a distal tip 136 of the cannula 134 has delivered at least one first amount of insulin. An adhesive patch 138 supports the cannula positioner body 120. The cannula positioner body 120 includes a position control member 142, which is longitudinally positioned in the positioner body and is slidable therein (e.g., the position control member 142 may be located beneath a slot 144 in the body. The position control member 142 may include one or more laterally projecting and elastically deflectable members 146 (e.g., elastically deflectable pawls). The position control member may be positioned within a track 148 or other like groove in the cannula positioner body 120, defined to include a plurality of teeth 150 or like projections. The teeth 150 may have a sloping surface 149 that allow the deflectable members to advance in one direction. The teeth 150 may have a blocking surface 151 that prevent motion in an opposite direction. In FIG. 2b, it can be seen how the length of the cannula may be adjusted to retract it, incrementally, in a longitudinal direction. As the position control member 142 is moved in a direction as indicated by the arrow, the elastically deflectable members 146 compress inwardly as they bear against the teeth 150 of the track 148. Once the deflectable members 146 pass a constricted region they elastically deform outwardly, and will resist moving in response to a force of being pulled in an opposite direction. Though not shown, it is possible in this specific embodiment, and in the other general embodiments to have a suitable mechanical restrictor that only allows a limited incremental advancement of the position control member and fixes the position once the position control member is in the desired location. For example, a protective cap, pin, clamp or the like may be positionable in the slot 144 (e.g., through a hole in both the position control member, and the positioner body) to fix the position between successive advancements. A laterally extending member may be employed to block movement of the position control member. In addition, or as an alternative, a protective cover or other suitable structure may be employed over the position control member 142 to limit access to the latter. As a result of the above, the position control member may be protected against accidental forces that may cause it to advance beyond an intended location. As seen, the cannula positioner body 120 is configured to matingly connect with a supply line connector (not shown) as previously described.

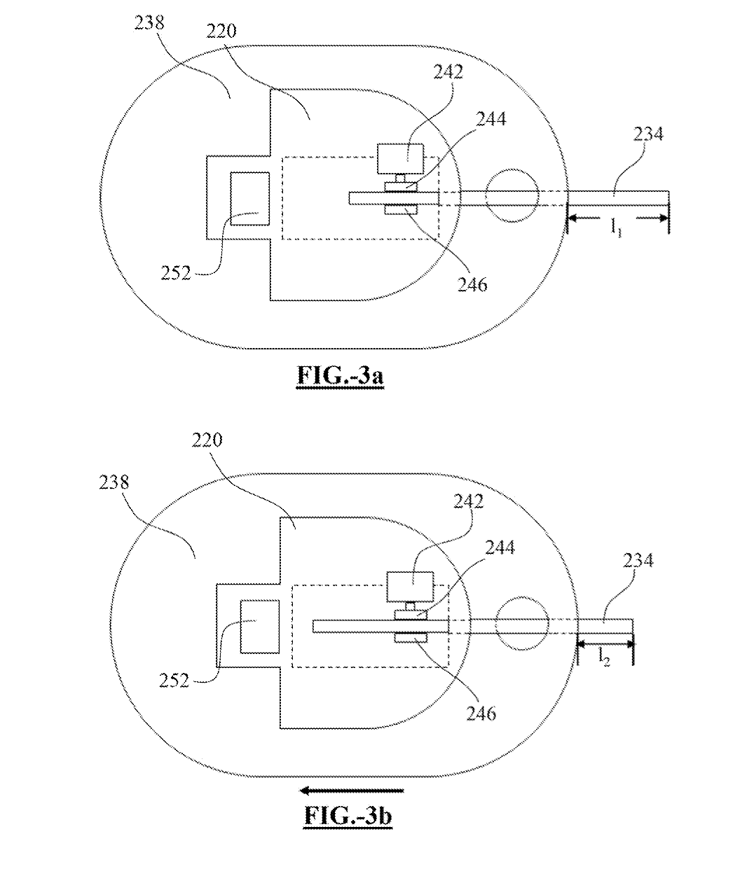

With reference to FIGS. 3a and 3b, there is seen another embodiment. In this embodiment, an adhesive patch 238 supports the cannula positioner body 220. A cannula positioner body 220 carries a rotary position control member 242. As can be appreciated, in general, the rotary position control member 242 may be employed for any of the embodiments taught herein, such as in FIGS. 2a and 2b. The rotary position control member 242 is adapted to be rotated in a manner so that it retracts a cannula 234. The rotary position control member 242 may have a gear and pawl configuration (not shown), or some other like structure, for incrementally translating the cannula 234. The rotary position control member 242 may act upon the cannula 234 by advancing the cannula 234 in a linear direction on the basis of rotary input. For example, the rotary position control member 242 may drive a roller 244 that urges the cannula 234 against an opposing surface 246 for causing the cannula 234 to linearly translate. In this embodiment, as in each other embodiment of the teachings generally, there may be an optional suitable cutter 252 adapted to cut one or more portions of the cannula 234 as its distal tip is retracted from an initial insulin delivery location.

As seen with reference to FIGS. 2a and 2b, and FIGS. 3a and 3b, upon retraction of the cannula during use, I₁ will be longer than I₂.

Also, it is seen that certain structures from the description of FIG. 1 have been omitted from the drawings of FIGS. 2a, 2b, 3a and 3b. However, such structures are contemplated as being a part of the structures shown in these drawings. For example the structures of each of FIGS. 2a, 2b, 3a and 3b may include a socket into which the flanking projections of a needle inserter or the line connector insert; they may include an arrangement for defining an interference fit with the line connector. For example, they may include a recess in the cannula positioner body so that an interference fit can be achieved (e.g., as between the bulbous portion of a latch finger and the forward facing wall). Accordingly, the various structural and co-acting features from FIG. 1 that are not currently shown in FIGS. 2a, 2b, 3a and 3b may, nonetheless, be employed in FIGS. 2a, 2b, 3a and 3. Likewise, features taught in e.g., FIGS. 6, 7 and 10, and associated written description of U.S. Pat. No. 6,056,718, incorporated by reference for all purposes; and FIGS. 1, 1a, 2, and 6-12, and associated written description of U.S. Pat. No. 7,303,543, incorporated by reference for all purposes, may also be employed.

As can be appreciated, in general, the cutter may be employed for any of the embodiments taught herein. The cutter may cut a portion of the cannula either at a portion located within or outside of the cannula positioner body. Any position control for a cannula may cause translation of the cannula distal tip in an indexed manner, such that each translation (e.g., translation) of the distal tip from an original location may be in a predetermined amount, and may be audibly, visually, and/or tactually sensed. The cannula may be a generally flexible tube (e.g., it may be a tube that can be bent at least about 60 degrees, 90 degrees or higher, without rupture, without kinking or both). The cannula may be a relatively rigid metal needle (e.g., a stainless steel needle). The supply line may be a polymeric tubing. Such polymeric tubing may have a length ranging from about 2 feet to about 4 feet. The cannula may be longer than conventional commercially available cannula (e.g., those available from Medtronic, Inc. under the name SILHOUETTE®) by at least about 1.3×, 1.6×, 2.0×, 3.0× or longer.

Use of the system of the present teachings can be performed with other tools in aid of installation. For example, insertion may be performed with an introducer needle tool such as one available from Medtronic, Inc., and or a Sil-Serter® insertion device such as available from Medtronic, Inc., either or both of which may be part of the aforenoted kit. Such tools may be modified to include a needle for guiding the cannula during insertion that has a length that is sufficiently long to extend to, and possibly also through, the distal tip of the cannula. See e.g., FIG. 5 (and associated written description) of U.S. Pat. No. 7,303,543, incorporated by reference for all purposes.

As can be seen, as to the specific embodiments and as to the general teachings, it is envisioned that the cannula of the present teachings will be disposed subcutaneously at one or more angles (α) that are less than about 75°, 60°, 45°, 30°, or even about 15° relative to a place that is tangent to the surface of a subject's skin at the point of insertion. It is possible that the cannula may include some or its entire portion at an angle (α) of about 90°. However, it is also possible that it may be free of (or substantially free of (e.g., less than about 70%, 80% or 90% of its subcutaneous length) any portion of the cannula that is disposed at an angle (α) of about 90°.

It will also be appreciated that male and female connector features depicted on one mating component of the devices described herein can be interchanged and employed with an opposing mating component. Further, though a well-known feature may not be described in detail in connection with one of the embodiments herein, or the general teachings, its use herein is contemplated nevertheless.

The teachings herein, in general, envision that the infusion set will have a generally low profile (e.g., it will have a maximum height relative to the skin surface to which it is inserted of less than about 25 mm, less than about 20 mm, less than about 15 mm). For example it may range from about 2 to about 15 mm, about 5 to about 12 mm or otherwise. The infusion set may be configured to provide a visual indicator that the components are connected, an audible indicator that the components are connected, or both. The device may be made of a polymeric material. For example, it may be made of an injection molded polymeric material. The device may be molded of separate parts that are attached together after molding. The device may be molded to include one or more parts that are molded in a clam-shell type configuration and folded over to assemble into a final configuration. The cannula may be flexible (e.g., it will bend without breaking when held at one of its ends). It may be rigid It may be a hollow polymeric tube. It may be a metallic tube. The device may include one or more protective covers over some or all of the device (e.g., to help prevent premature or accidental movement of the cannula positioner.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30” inclusive of at least the specified endpoints.

The disclosures of all articles and references, including patent applications and publications, are incorporated by reference, for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consisting of, the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

Relative positional relationships of elements depicted in the drawings are part of the teachings herein, even if not verbally described. Further, geometries shown in the drawings (though not intended to be limiting) are also within the scope of the teachings, even if not verbally described.

What is claimed is:

1. A device for introducing liquid into tissue of a subject in need of the liquid. comprising:
(a) a cannula having a distal tip;
(b) a cannula positioner adapted for adjustably controlling a position of the cannula after introduction subcutaneously into a subject;
(c) a cannula positioner body configured to allow adjustable support of the cannula and the cannula positioner and being wearable by the subject; and
wherein the cannula positioner includes a cannula position control member that is adapted to be driven manually, in an automated manner or both, to incrementally advance or retract the cannula so that a subcutaneous position of the distal tip is changeable to one or more different subcutaneous positions over a period of at least four days, without removal of the cannula from the subject.

2. The device of claim 1, wherein the device includes an interface structure operably connectable with and disconnectable from a liquid supply device in a manner to introduce a liquid, when connected with the liquid supply device, from a supply of the liquid associated with one or both of the liquid supply device, or the cannula positioner body to the cannula, and into the subject.

3. The device of claim 2, wherein t cannula positioner body carries a source of liquid in liquid communication with the cannula.

4. The device of claim 3, wherein the cannula positioner body carries a pump that is operative to cause delivery of the liquid to the cannula for introduction into the subject.

5. The device of claim 1, wherein the cannula has a length of about 20 mm to about 50 mm that is subcutaneously insertable the extent of the length to a first subcutaneous position to which a liquid is delivered and is operable for periodic and incremental removal from the first subcutaneous position.

6. The device of claim 5, wherein the cannula positioner, the cannula positioner body or both include a structure defining a guide track along which the cannula positioner is translatable so that the cannula is able to be repositioned subcutaneously relative to the first subcutaneous position to one or more second subcutaneous positions.

7. The device of claim 6, wherein the cannula positioner includes a knob that is configured to be manually actuated to cause the cannula to be repositioned subcutaneously relative to the first subcutaneous position to the one or more second subcutaneous positions.

8. The device of claim 7, wherein a manual actuation of the knob is in a rotary direction that causes at least a portion of the cannula positioner to translate the cannula generally longitudinally between two or more subcutaneous positions.

9. The device of claim 8, wherein the manual actuation of the knob is in a longitudinal axial direction that causes at least a portion of the cannula positioner to translate the cannula generally longitudinally between the two or more subcutaneous positions.

10. The device of claim 6, wherein the cannula positioner includes a direction restrictor that is configured and operable to permit the cannula to move from a first subcutaneous position to one or more second subcutaneous positions, while preventing the cannula from returning toward the first subcutaneous position from the one or more second subcutaneous positions.

11. The device of claim 10, wherein the direction restrictor includes one or more mechanical devices adapted to allow movement of the cannula in at least one retracted direction while preventing movement of the cannula in an insertion direction relative to the subject.

12. The device of claim 3, wherein the cannula positioner body is adapted to be mechanically connected with a conduit associated with a liquid supply control unit.

13. The device of claim 12, wherein the cannula positioner body includes one or more receptacles into which the liquid is able to be introduced by way of the conduit.

14. A method of using the device of claim for administering a liquid to a mammal ire need of the liquid, comprising inserting the device into the mammal to locate the cannula in a first subcutaneous position, and periodically and incrementally changing the location of the cannula relative to the first subcutaneous position, while maintaining the cannula within the mammal continuously for a period of at least four consecutive days.

15. The method of claim 14, wherein the method includes actuating a cannula positioner to incrementally change the location of the cannula relative to the first subcutaneous position.

16. The method of claim 15, wherein the cannula positioner includes a knob which when actuated in a rotary direction causes at least a portion of the cannula positioner to translate the cannula longitudinally between two or more subcutaneous positions.

17. The method of claim 16, wherein the method includes placing the device n fluid communication with a liquid supply.

18. The method of claim 17, wherein the liquid administered to the mammal includes insulin.

19. A kit comprising the device of claim 1, wherein the kit includes one or more of a pump, a liquid delivery tube, a reservoir containing a liquid for introduction to a subject in need of the liquid, a skin preparation composition, a skin barrier, an adhesive remover, a topical medicament, a tape, a dressing, a casing, or any other accessory for enhancing the comfort and/or longevity of wear for the subject.

20. A device for introducing insulin into tissue of a subject in need of the insulin, comprising:
(a) a cannula having a distal tip and a length of about 20 mm to about 50 mm, wherein the cannula is subcutaneously insertable to a first subcutaneous position to which the insulin is delivered;
(b) a cannula positioner which adjustably controls a position of the cannula after introduction subcutaneously into the subject, the cannula positioner including:
(i) a cannula position control member which is able to be driven to incrementally advance or retract the cannula;
(ii) a manually actuated knob which drives the cannula position control member;
(c) a cannula positioner body which allows adjustable support of the cannula and the cannula positioner and is adapted to connect to a fluid supply line connector; the cannula positioner body including:
(i) one or more female portions adapted to matingly connect with one or more male portions of the fluid supply line connector;
(ii) a receptacle which is adapted to receive a needle of the fluid supply line connector to receive the insulin;
(iii) a guide track along which the cannula position control member is translatable so that the cannula is able to be repositioned subcutaneously relative to the first subcutaneous position to one or more second subcutaneous positions;
(d) an adhesive patch connected to the cannula positioner body so that both the cannula positioner body and the adhesive patch are wearable by the subject and the adhesive patch includes a throughhole which the cannula penetrates; and
wherein when the cannula position control member is driven by the manually actuated knob, the cannula position control member incrementally advances or retracts the cannula so that a subcutaneous position of the distal tip is changeable to one or more different subcutaneous positions over a period of at least four days, without removal of the cannula from a subject.
